(19) United States
(12) Patent Application Publication
(10) Pub. No.: US 2014/0199382 A1
(43) Pub. Date: Jul. 17, 2014

(54) STABLE PHARMACEUTICAL COMPOSITIONS OF AN S1P RECEPTOR AGONIST

(71) Applicant: Cadila Healthcare Limited, Gujarat (IN)

(72) Inventors: SUSHRUT KRISHNAJI KULKARNI, Gujarat (IN); AJAYKUMAR HANDA, Gujarat (IN); PRUDHVI RAJU DANTULURI, Gujarat (IN)

(73) Assignee: Cadila Healthcare Limited, Gujarat (IN)

(21) Appl. No.: 14/152,777

(22) Filed: Jan. 10, 2014

(30) Foreign Application Priority Data
Jan. 11, 2013 (IN) 103/MUM/2013

(51) Int. Cl. A61K 31/138

(52) U.S. Cl. CPC A61K 31/138
USPC 424/451; 514/653

(57) ABSTRACT

The present invention relates to a stable pharmaceutical composition comprising an S1P receptor agonist and one or more pharmaceutically acceptable excipients, wherein the composition is free of a sugar alcohol. It also relates to method of preparing such compositions and using those compositions in the treatment of multiple sclerosis.

### FIELD OF THE INVENTION

The present invention relates to a stable pharmaceutical composition comprising an S1P receptor agonist and one or more pharmaceutically acceptable excipients, wherein the composition is free of a sugar alcohol. It also relates to method of preparing such compositions and using those compositions in the treatment of multiple sclerosis.

### BACKGROUND OF THE INVENTION

The present invention relates to pharmaceutical compositions comprising a sphingosine-1 phosphate receptor agonist. Sphingosine-1 phosphate (hereinafter “S1P”) is a natural serum lipid. Presently there are 8 known S1P receptors, namely S1P1 to S1P8. S1P receptor agonists have accelerating lymphocyte homing properties.

S1P receptor agonists are immunomodulating compounds which elicit a lymphopenia resulting from a re-distribution, preferably reversible, of lymphocytes from circulation to secondary lymphatic tissue, evoking a generalized immunosuppression. Naive cells are sequestered, CD4 and CD8 T-cells and B-cells from the blood are stimulated to migrate into lymph nodes (LN) and Peyer's patches (PP), and thus infiltration of cells into transplanted organs is inhibited.

The various known S1P receptor agonists show structural similarities, which result in related problems in providing a suitable formulation. In particular, there is a need for an S1P receptor agonist containing formulation which is well-adapted for oral administration in a solid form, e.g. as a tablet or a capsule.

A preferred S1P receptor agonist in the present invention is 2-amino-2-tetradecyl-1,3-propanediol. A particularly preferred S1P receptor agonist is fingolimod (FTY720), i.e. 2-amino-2-[2-(4-octylphenyl)ethyl]propane-1,3-dial in free form or in a pharmaceutically acceptable salt form, e.g. the hydrochloride, as shown:

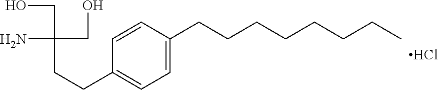 [SMILES: CCCCCCCCc1ccc(CCC(N)(CO)CO)cc1 ; Cl]

This particular compound is disclosed in U.S. Pat. No. 5,604,229.

U.S. patent application 20110105620 discloses a solid pharmaceutical composition suitable for oral administration, comprising: (a) a S1P receptor agonist; and (b) a sugar alcohol. It discloses compositions of compound FTY720 and a sugar alcohol selected from mannitol, maltitol, inositol, xylitol or lactitol. It discloses that solid compositions comprising a sugar alcohol are particularly well suited to the oral administration of S1P receptor agonists.

U.S. patent application 20100040678 discloses an oral pharmaceutical composition comprising an S1P receptor modulator, wherein the composition comprises a coating comprising: (a) one or more polymer resins (b) one or more metal oxides. It also discloses the composition comprising an S1P receptor modulator and microcrystalline cellulose in the absence of a sugar alcohol.

U.S. patent application 20100267675 discloses a stable pharmaceutical composition of an S1P receptor modulator and various excipients. It discloses stability studies of various excipients with fingolimod.

PCT application 20110131368 discloses a method of preparing an intermediate containing fingolimod, a method of preparing granules containing fingolimod, a method of preparing an oral dosage form containing fingolimod and accordingly intermediates, granules and oral dosage forms obtainable by that method. It discloses the composition wherein joint comminution is carried out in such a way that the resulting intermediate particles have a specific particle size distribution to achieve homogeneity in the product.

There is still a long and unmet need to prepare a stable composition of a S1P receptor agonist without using a sugar alcohol as disclosed in the art. Accordingly, the present Invention provides a solid pharmaceutical composition suitable for oral administration, comprising a S1P receptor agonist and a suitable excipient other than a sugar alcohol.

The compositions provide a convenient means of systemic administration of S1P receptor agonists, do not suffer from the disadvantages of liquid formulations for injection or oral use, and have good physicochemical and storage properties. In particular, the compositions of the present invention may show a high level of uniformity in the distribution of the S1P receptor agonist throughout the composition, as well as high stability.

In the present invention, we have discovered that pharmaceutical compositions, when prepared without using any sugar alcohol are stable and showed a dissolution profile comparable to Gilenya®.

### SUMMARY OF THE INVENTION

In one general aspect, there is provided a stable pharmaceutical composition comprising an S1P receptor agonist and one or more pharmaceutically acceptable excipients, wherein the composition is free of a sugar alcohol.

In another general aspect, there is provided a stable pharmaceutical composition comprising an S1P receptor agonist and pregelatinized starch, wherein the composition is free of a sugar alcohol.

In another general aspect, there is provided a stable pharmaceutical composition comprising fingolimod hydrochloride and pregelatinized starch, wherein the composition is free of a sugar alcohol.

In another general aspect, there is provided a stable pharmaceutical composition comprising fingolimod hydrochloride and low-substituted hydroxypropylcellulose, wherein the composition is free of a sugar alcohol.

In another general aspect, there is provided a stable pharmaceutical composition comprising fingolimod hydrochloride and dibasic calcium phosphate, wherein the composition is free of a sugar alcohol.

In another general aspect, there is provided a stable pharmaceutical composition comprising fingolimod or pharmaceutically acceptable salt thereof and one or more pharmaceutically acceptable excipients, wherein the excipients are selected from one or more of starch, pregelatinized starch, powdered cellulose, dibasic calcium phosphate, tribasic calcium phosphate, citric acid, tartaric acid, maleic acid, sodium lauryl sulphate, polysorbate, poloxamer or calcium silicate.

Embodiments of the pharmaceutical composition may include one or more of the following features. For example, the pharmaceutical composition may further include one or more pharmaceutically acceptable excipients. The pharmaceutically acceptable excipients may include one or more diluents, disintegrants, binders, lubricants, glidants, acidifiers, surfactants, solvents and the like.

In another general aspect, there is provided a process for preparing a pharmaceutical composition of an S1P receptor agonist, wherein the composition does not contain any sugar alcohol. The process includes a step of mixing and/or granulating an S1P receptor agonist with one or more pharmaceutically acceptable excipients and converting the mixture thus obtained into suitable pharmaceutical dosage form.

In another general aspect, there is provided a process for preparing a pharmaceutical composition of fingolimod, wherein the composition does not contain any sugar alcohol. The process includes a step of mixing and/or granulating fingolimod with one or more pharmaceutically acceptable excipients and converting the mixture thus obtained into a suitable pharmaceutical dosage form.

In another general aspect, there is provided a stable pharmaceutical composition comprising fingolimod or salts thereof, wherein the composition retains at least 80% of potency of fingolimod or salts thereof when stored in the conditions as per ICH guidelines, characterized in that said composition does not contain any sugar alcohol.

In another general aspect, there is provided a stable pharmaceutical composition of fingolimod, wherein the composition is free of any sugar alcohol, and the composition exhibits no significant difference in rate and/or extent of absorption of fingolimod as compared to marketed formulation of fingolimod available under the trade name Gilenya®.

The details of one or more embodiments of the invention are set forth in the description below. Other features, objects and advantages of the invention will be apparent from the description.

### DETAILED DESCRIPTION OF THE INVENTION

The inventors of the invention have discovered that when compositions of fingolimod are prepared without using any sugar alcohol, these compositions still exhibit better or at least same bioavailability as compared to the capsules marketed under the trade name Gilenya® which contains mannitol as a sugar alcohol. The inventors have noticed that by judicial selection of excipients in its optimum concentrations, and particularly without using any additional sugar alcohol, the stable formulations can be prepared with good physicochemical and storage properties.

The term “fingolimod” used throughout the specification refers to not only fingolimod per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. Examples of pharmaceutically acceptable salts of the compounds of present invention include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals, such as sodium, potassium, calcium and aluminium, salts with amines, such as triethylamine and salts with dibasic amino acids, such as lysine. Preferred salt of fingolimod is its hydrochloride.

The term “stable composition” used throughout the specification refers to a stable composition which shows no more than 1% of any single unknown impurity upon storage for at least 3 months under 30° C./40° C. and 75% RH.

The pharmaceutical compositions as described herein may be prepared by processes known to the person having ordinary skill in the art of pharmaceutical technology such as direct filling, wet granulation, dry granulation or melt granulation.

The pharmaceutically acceptable excipients may include one or more fillers, disintegrants, binders, lubricants, glidants, acidifiers, surfactants and the like.

Suitable diluents or fillers may include, but are not limited to, starch, pregelatinized starch, anhydrous dibasic calcium phosphate, tribasic calcium phosphate, dihydrate dibasic calcium phosphate, low-substituted hydroxypropylcellulose, powdered cellulose, microcrystalline cellulose, calcium silicate, calcium phosphate, calcium carbonate, magnesium carbonate, magnesium oxide, calcium sulphate, hydrogenated vegetable oil, dextrin, cyclodextrin, kaolin and the like. The diluents are devoid of any sugar alcohol like lactose, sucrose, dextrose, mannitol or sorbitol, maltitol, inositol, xylitol or lactitol.

Suitable binders may include, but not limited to one or more of methyl cellulose, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums, synthetic resins and the like.

Suitable disintegrants may include, but are not limited to, croscarmellose sodium, crospovidone, sodium starch glycolate, corn starch, potato starch, maize starch and modified starches, calcium silicates, low substituted hydroxypropylcellulose. The amount of disintegrating agent is preferably in the range of 5% to 35% by weight of the composition.

Suitable lubricants and/or glidants may include, but are not limited to, stearic acid and its derivatives or esters like sodium stearate, magnesium stearate and calcium stearate and the corresponding esters such as sodium stearyl fumarate; talc and colloidal silicon dioxide respectively. The amount of lubricant and/or glidant is preferably in the range of 0.25% to 5% by weight of the composition.

As acidifiers it is common to use substances which, when dissolved in water, lead to a pH of less than 7.0. Acidifiers are preferably compounds, especially organic compounds, which have at least one acid group. The compounds may be present as the free acid or the salt. In the case of salts, alkaline or alkaline earth salts are preferred, especially sodium or potassium salts. Suitable acidifiers may include, but are not limited to, adipic acid, malic acid, ascorbic acid, succinic acid, citric acid, fumaric acid, glutaric acid, maleic acid, malonic acid, tartaric acid and/or salts thereof.

Suitable surfactants may include, but are not limited to, anionic, cationic, non-ionic or amphoteric surfactants or those known to the person skilled in the art. Suitable surfactants include mono fatty acid esters of polyoxyethylene sorbitan such as those sold under the brand name Tween®; sodium lauryl sulfate, polyoxyethylene castor oil derivatives such as those sold under the brand name Cremophor®, polyethoxylated fatty acids and their derivatives, propylene glycol fatty acid esters, sterol and sterol derivatives; sorbitan fatty acid esters and their derivatives, sugar esters, polyoxyethylene-polyoxypropylene block copolymers such as those sold under the brand name Poloxamer, soy lecithin, sodium stearyl fumarate, and the like. The amount of surfactant is preferably in the range of 0.5% to 25% by weight of the composition.

In one embodiment, the stable compositions may be prepared by mixing and/or granulating an S1P receptor agonist with one or more pharmaceutically acceptable excipients and converting the mixture thus obtained into suitable pharmaceutical dosage form, wherein the excipients are devoid of any sugar alcohol.

In another embodiment, capsules may be prepared by mixing fingolimod hydrochloride with pregelatinized starch and lubricating the mixture and filling the lubricated mixture in to the capsules. Pregelatinized starch used herein may be replaced by dibasic calcium phosphate and low-substituted hydroxypropylcellulose.

In another embodiment, capsules may be prepared by granulating fingolimod hydrochloride, pregelatinized starch, and other pharmaceutically acceptable excipients with the binder solution; drying the granules; lubricating the granules and filling the lubricated granules in to the capsules.

As per the preferred embodiment of the present invention, particle size of the active ingredient used in this invention is such that d₉₀ is less than 150 microns, preferably less than 50 microns and d₅₀ is less than 75 microns, preferably less than 25 microns. Active ingredient and the pharmaceutically acceptable excipients may be mixed and milled together to give the preferred particle size distribution of the mixture. This specific particle size distribution of the drug or the mixture is favorable as to achieve content uniformity of the final dosage form.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLES

### Example 1

| | | Quantity |
|---|---|---|
| Sr. No | Ingredient | (mg/capsule) |
| 1 | Fingolimod hydrochloride | 0.56 |
| 2 | Pregelatinized Starch (Starch 1500) | 75.04 |
| 3 | Sodium starch glycolate | 4.00 |
| 4 | Sodium stearyl fumarate | 0.40 |
| 5 | Hard gelatin Capsule | |
| | Total | 80.00 |

### Process:

Fingolimod, pregelatinized starch and sodium starch glycolate were geometrically mixed and passed through sieve. The mixture was lubricated with sodium stearyl fumarate and filled into capsules.

### Stability Study of Example 1:

| | | 30° C./75% RH, | 40° C./75% RH, |
|---|---|---|---|
| Impurity | Initial | 15 days | 15 days |
| Coupled ketone | ND | ND | ND |
| Acetyl fingolimod | ND | ND | ND |
| Diester | ND | ND | ND |
| Single individual | 0.07 | 0.04 | 0.11 |
| unknown | | | |
| Total | 0.07 | 0.04 | 0.17 |

### Example 2

| | | Quantity |
|---|---|---|
| Sr. No | Ingredient | (mg/capsule) |
| 1 | Fingolimod hydrochloride | 0.56 |
| 2 | Dibasic calcium phosphate, anhydrous | 71.04 |
| 3 | Sodium starch glycolate | 4.00 |
| 4 | Sodium stearyl fumarate | 0.40 |
| 4 | Hard gelatin Capsule | |
| | Total | 80.00 |

### Process:

Fingolimod, anhydrous dibasic calcium phosphate and sodium starch glycolate were geometrically mixed and passed through sieve. The mixture was lubricated with sodium stearyl fumarate and filled into capsules.

### Stability Study of Example 2:

| Impurity | Initial | 40° C./75% RH, 15 days |
|---|---|---|
| Coupled ketone | ND | ND |
| Acetyl fingolimod | ND | ND |
| Diester | ND | ND |
| Single individual | 0.03 | 0.06 |
| unknown | | |
| Total | 0.03 | 0.06 |

### Example 3

| | | Quantity |
|---|---|---|
| Sr. No | Ingredient | (mg/capsule) |
| 1 | Fingolimod hydrochloride | 0.56 |
| 2 | Low-substituted | 48.44 |
| | hydroxypropylcellulose (L-HPC) | |
| 3 | Magnesium Stearate | 1.00 |
| 4 | Hard gelatin Capsule | |
| | Total | 50.00 |

### Process:

Fingolimod and Low-substituted hydroxypropyl cellulose (L-HPC) were geometrically mixed and passed through sieve. The mixture was lubricated with magnesium stearate and filled into capsules.

### Example 4

| | | Quantity |
|---|---|---|
| Sr. No | Ingredient | (mg/capsule) |
| 1 | Fingolimod hydrochloride | 0.56 |
| 2 | Pregelatinized Starch (Starch 1500) | 24.22 |
| 3 | Dibasic calcium phosphate | 24.22 |
| 4 | Magnesium Stearate | 1.00 |
| 5 | Hard gelatin Capsule | |
| | Total | 50.00 |

### Process:

Fingolimod, dibasic calcium phosphate and pregelatinized starch were geometrically mixed and passed through sieve. The mixture was lubricated with magnesium stearate and filled into capsules.

### Example 5

| | | Quantity |
|---|---|---|
| Sr. No | Ingredient | (mg/capsule) |
| 1 | Fingolimod hydrochloride | 0.56 |
| 2 | Low-substituted | 24.22 |
| | hydroxypropylcellulose (L-HPC) | |
| 3 | Dibasic calcium phosphate | 24.22 |
| 4 | Magnesium Stearate | 1.00 |
| 5 | Hard gelatin Capsule | |
| | Total | 50.00 |

### Process:

Fingolimod, low-substituted hydroxypropylcellulose (L-HPC) and dibasic calcium phosphate were geometrically mixed and passed through sieve. The mixture was lubricated with magnesium stearate and filled into capsules.

### Example 6

| | | Quantity |
|---|---|---|
| Sr. No | Ingredient | (mg/capsule) |
| 1 | Fingolimod hydrochloride | 0.56 |
| 2 | Pregelatinized Starch (Starch 1500) | 24.22 |
| 3 | Low-substituted | 12.11 |
| | hydroxypropylcellulose (L-HPC) | |
| 4 | Dibasic calcium phosphate | 12.11 |
| 5 | Magnesium Stearate | 1.00 |
| 6 | Hard gelatin Capsule | |
| | Total | 50.00 |

### Process:

Fingolimod, low-substituted hydroxypropylcellulose (L-HPC), dibasic calcium phosphate and pregelatinized starch were geometrically mixed and passed through sieve. The mixture was lubricated with magnesium stearate and filled into capsules.

### Example 7

| Sr. No | Ingredient | Quantity (% w/w) |
|---|---|---|
| 1 | Fingolimod hydrochloride | 1%-5% |
| 2 | Powdered Cellulose | 10%-50% |
| 3 | Pregelatinized starch | 10%-50% |
| 4 | Purified water | q.s. |
| 5 | Talc | 1%-10% |
| 6 | Magnesium stearate | 0.5-5% |
| 7 | Hard gelatin Capsule | |

### Process:

Fingolimod hydrochloride, powdered cellulose, pregelatinized starch were geometrically mixed, passed through sieve and granulated with purified water. The granules were dried and lubricated with talc and magnesium stearate. This lubricated mass was filled into capsules.

### Example 8

| Sr. No | Ingredient | Quantity (% w/w) |
|---|---|---|
| 1 | Fingolimod hydrochloride | 1%-5% |
| 2 | Dibasic Calcium phosphate | 60%-98% |
| 3 | Sodium starch glycolate | 2-10% |
| 4 | Purified water | q.s. |
| 6 | Talc | 1%-10% |
| 7 | Sodium stearyl fumarate | 0.5-5% |
| 8 | Hard gelatin Capsule | |

### Process:

Fingolimod hydrochloride, dibasic calcium phosphate and sodium starch glycolate were geometrically mixed, passed through sieve and granulated with purified water. The granules were dried, mixed with remaining quantity of sodium starch glycolate and lubricated with talc and sodium starch glycolate. This lubricated mass was filled into capsules.

### Example 9

| Sr. No | Ingredient | Quantity (% w/w) |
|---|---|---|
| 1 | Fingolimod hydrochloride | 1%-5% |
| 2 | Pregelatinized starch | 30%-50% |
| 3 | Hydroxypropylcellulose | 1-5% |
| 4 | Citric acid | 1-10% |
| 5 | Isopropyl alcohol | q.s. |
| 6 | Crospovidone | 2-10% |
| 7 | Talc | 1%-10% |
| 8 | Magnesium stearate | 0.5-5% |
| 9 | Hard gelatin Capsule | |

### Process:

Fingolimod hydrochloride and pregelatinized starch were mixed together to form a mixture. Hydroxypropylcellulose and citric acid were dissolved in isopropyl alcohol and the above mixture was granulated with this binder solution. The granules were dried, mixed with crospovidone and talc and lubricated with magnesium stearate. This lubricated mass was filled into capsules.

### Example 10

| Sr. No | Ingredient | Quantity (% w/w) |
|---|---|---|
| 1 | Fingolimod hydrochloride | 1%-5% |
| 2 | Starch | 30%-50% |
| 3 | Povidone | 1-5% |
| 4 | Poloxamer | 1-10% |
| 5 | Purified water | q.s. |
| 6 | Croscarmellose sodium | 2-10% |
| 7 | Colloidal silicon dioxide | 1%-10% |
| 8 | Magnesium stearate | 0.5-5% |
| 9 | Hard gelatin Capsule | |

### Process:

Fingolimod hydrochloride and starch were mixed together to form a mixture. Povidone and poloxamer were dissolved in purified water and the above mixture was granulated with this binder solution. The granules were dried, mixed with croscarmellose sodium and lubricated with colloidal silicon dioxide and magnesium stearate. This lubricated mass was filled into capsules.

We claim:

1. A stable pharmaceutical composition comprising an S1P receptor agonist and one or more pharmaceutically acceptable excipients, wherein the composition is free of a sugar alcohol.

2. The stable composition according to claim 1, wherein the S1P receptor agonist is 2-amino-2-[2-(4-octylphenyl)ethyl]propane-1,3-diol or pharmaceutically acceptable salt thereof.

3. The stable composition according to claim 1 comprising 0.01 to 5% by weight of the S1P receptor agonist.

4. The stable composition according to claim 1, wherein the S1P receptor agonist is micronized such that d₉₀ is less than 50 microns and d₅₀ is less than 25 microns.

5. The stable composition according to claim 1, wherein the excipients comprise one or more of diluents, disintegrants, binders, lubricants, glidants, acidifiers, surfactants and solvents.

6. The stable composition according to claim 1, wherein the excipients comprise one or more of starch, pregelatinized starch, powdered cellulose, dibasic calcium phosphate, tribasic calcium phosphate, citric acid, tartaric acid, fumaric acid, maleic acid, sodium lauryl sulphate, polysorbate, poloxamer or calcium silicate.

7. The stable composition according to claim 1, wherein the excipient is sodium starch glycolate.

8. The stable composition according to claim 1, wherein the excipient is dibasic calcium phosphate.

9. The stable composition according to claim 8, wherein dibasic calcium phosphate is present in an amount of more than 80% by weight of the composition.

10. The stable composition according to claim 1, wherein the composition is in the form of capsules.

11. The stable composition according to claim 1, wherein the composition shows no more than 1% of any individual impurity.

12. A process for preparing the stable composition of claim 1, wherein the process comprises the steps of:
mixing and/or granulating the S1P receptor agonist and one or more excipients;
drying the granules;
lubricating the mixture/granules; and
filling in to the capsules.

13. The composition according to any proceeding claims, wherein the composition is used for the treatment of multiple sclerosis.
